(19) United States
(12) Patent Application Publication
(10) Pub. No.: US 2012/0209200 A1
(43) Pub. Date: Aug. 16, 2012

(54) Drive Assembly for a Pen-Type Injector and Pen-Type Injector with a Drive Assembly

(75) Inventors: Mathew Jones, Warwick (GB); Christopher Jones, Tewkesbury (GB); Christopher Langley, Leamington Spa (GB)

(73) Assignee: SANOFI-AVENTIS DEUTSCHLAND GMBH, Frankfurt am Main (DE)

(21) Appl. No.: 13/383,984

(22) PCT Filed: Jul. 14, 2010
(86) PCT No.: PCT/EP2010/060128

§ 371 Date: May 7, 2012

(30) Foreign Application Priority Data
Jul. 15, 2009 (EP) 09009215.6

(51) Int. Cl. A61M 5/20

(52) U.S. Cl. 604/155

(57) ABSTRACT

A drive assembly (2) for a pen-type injector, comprising: a guiding means (40) having a first (42) and a second end (41), a drive means (20), an elongate member (30) coupled to the guiding means (40) and adapted to be driven by the drive means (20) for displacing the guiding means (40) with respect to the drive means (20), a piston member (60) displaceably disposed at the first end (42) of the guiding means (40) and an adjusting member (62) displaceably disposed at the second end (41) of the guiding means (40), wherein the drive means (20) is fixedly located with respect to the adjusting member (62), and wherein the piston member (60) and the adjusting member (62) are coupled such that the sum of the distance between the piston member (60) and the guiding means (40) and the distance between the adjusting means (62) and the guiding means (40) remains constant, and a pen-type injector with such a drive assembly.

The present invention relates to a drive assembly for a pen-type injector and a pen-type injector with such a drive assembly.

In particular, the present invention relates to pen-type injectors, that is to injectors of the kind that provide for administration by injection of medicinal products from a multidose cartridge. In particular, the present invention also relates to such injectors where a user may set the dose.

Such injectors have application where regular injection by persons without formal medical training occurs. This is increasingly common amongst those having diabetes where self-treatment enables such persons to conduct effective management of their diabetes.

These circumstances imply a number of requirements for pen-type injectors of this kind. The injector must be robust in construction, yet easy to use both in terms of the manipulation of the parts and understanding by a user of its operation. In the case of those with diabetes, many users will be physically infirm and may also have impaired vision.

Multidose-cartridge pen-type injectors providing for administration by injection of medicinal products from a multidose cartridge are for example generally known from CA 02231481 and CA 02184579 which both disclose a multi-processor controlled, spring-biased, single piston drive assembly for a pen-type injector.

Document DE 20 2004 020 726 U1 discloses a hydraulic dosing device with three or more syringes. The syringes are coupled to each other mechanical or by fluid communication lines. Due to the activation of the device by an extra syringe and the coupling of such syringe via fluid communication lines, the whole device of this document is relatively big and not useful for incorporation in pen-type injectors.

In the European application EP 1 754 498 A1 a driving device for injection or infusion devices is disclosed. Such device is very complex, in particular with respect to a pushing means, which has to be advanced around a corner.

DE 0 2004 052 628 A1 discloses the filling or the refilling of an injection cartridge. Such a system is not able to offer a simple and light weight solution which is also very compact, for expelling fluid out of the cartridge.

Generally, multidose cartridges have a bung or piston at one end that is driven towards a second end of the cartridge to expel the medical product from the pen-type injector. It is a problem that pen-type injectors should be small enough to unobtrusively fit into a jacket pocket or a hand bag without difficulty. At the same time, the pen-type injector must be of a size that enables a piston or the like used to drive the cartridge bung within the cartridge to be moved both to a maximum dispense position within the cartridge and to be fully withdrawn from the cartridge to allow for replacement of the cartridge.

It is an objective of the present invention to provide an improved drive assembly for a pen-type injector and improved pen-type injector in line with these differing requirements.

In order to achieve this objective, the present invention provides a drive assembly for a pen-type injector according to claim 1 and a pen-type injector according to claim 12. The dependent claims which are referred back to these claims relate to advantageous embodiments of the present invention.

According to an aspect of the invention, a drive assembly for a pen-type injector is provided, comprising: a guiding means having a first and a second end, a drive means, an elongate member coupled to the guiding means and adapted to be driven by the drive means for displacing the guiding means with respect to the drive means, a piston member displaceably disposed at the first end of the guiding means and an adjusting member displaceably disposed at the second end of the guiding means. The drive means is fixedly located with respect to the adjusting member. Further, the piston member and the adjusting member are coupled such that the sum of the distance between the piston member and the guiding means and the distance between the adjusting means and the guiding means remains constant.

In the drive assembly according to the invention, the guiding means can be formed as a U-shaped device.

According to an example of the invention, the elongate member can be adapted to be rotated by the drive means and wherein the guiding means comprises an opening, the opening and the elongate member forming a threaded connection between the U-shaped guiding means and the elongate member, rotation of the elongate member causing the housing to process along the elongate member, wherein the adjusting member is fixedly located with respect to the elongate member.

According to an example of the invention, the guiding means can be designed such that is forms a housing with a passageway, wherein the piston member is displaceable located within a first end of the passageway, wherein the adjusting member is formed as a second piston member and is displaceable located within a second end (41) of the passageway. In this regard, the housing can be filled with an incompressible medium so that the housing, the incompressible medium and the first piston member and the second piston member are configured such that a displacement of the second piston member with regard to the passageway causes a displacement of the first piston member. Further, the passageway can be designed such that it defines a first passage portion, a second passage portion extending parallel to the first passage portion and an intermediate passage portion connecting the first passage portion to the second passage portion.

According to an example of the invention, the drive means comprises a motor and a gear train.

According to an example of the invention, the elongate member is provided with a helical rib to engage a helical thread provided in an opening in the housing.

According to an example of the invention, the drive means comprises a motor drivingly connected to a gear train for driving the elongate member. The motor can be a stepper motor. Further, the motor can be micro-processor controlled.

According to another aspect of the invention, a drive assembly for a pen-type injector is provided, comprising a drive means, an elongate member adapted to be rotated by the drive means, a housing having an opening through which the elongate member is threadedly connected to the housing and a passageway defined within the housing, rotation of the elongate member causing the housing to process along the elongate member, a first piston member displaceably located within a first end of the passageway and a second piston member displaceably located within a second end of the passageway and fixedly located with respect to the elongate member. Preferably the pen-type injector or the injector further comprises a replaceable cartridge containing medicinal product.

In these embodiments of the invention, the drive means can generally comprise a motor drivingly connected to a gear train.

In these embodiments of the invention, the passageway can generally be defined by a first passage portion, a second passage portion extending substantially parallel to the first passage portion and an intermediate passage portion connecting the first passage portion to the second passage portion.

Preferably, the elongate member is provided with a helical rib to engage a helical thread provided in an opening in the housing.

In accordance with a second aspect of the invention, a pen-type injector comprises a main housing, a power source and a drive mechanism according to the first aspect of the present invention.

According to another aspect of the invention, the pen-type injector comprises a main housing, and a drive assembly according to any previous claim and a power source for supplying the drive mechanism, wherein the guiding means which is moveably integrated within the main housing. In this regard, the injector can comprise a replaceable cartridge, wherein the cartridge can be fixedly located with respect to the elongate member, wherein the guiding means is disposed displaceable with regard to the cartridge and wherein the guiding means is configured such that at least in an extended position of the same the first end of the guiding means extends into one end of the cartridge for dispensing a medium contained in the cartridge when the guiding means is moved into a dispensing direction.

The term “housing” according to instant invention shall preferably mean any exterior housing (“main housing”, “body”, “shell”) or interior housing (“insert”, “inner body”) having one or more helical threads. The housing may be designed to enable the safe, correct, and comfortable handling of the drug delivery device or any of its mechanism. Usually, it is designed to house, fix, protect, guide, and/or engage with any of the inner components of the drug delivery device (e.g., the drive mechanism, cartridge, plunger, piston rod) by limiting the exposure to contaminants, such as liquid, dust, dirt etc. In general, the housing may be unitary or a multipart component of tubular or non-tubular shape. Usually, the exterior housing serves to house a cartridge, which may be replaceable or non-replaceable, from which a number of doses of a medicinal product may by dispensed. In a more specific embodiment of instant invention, the housing is provided with a plurality of maximum dose stops adapted to be abutted by a radial and/or axial stop provided on the activation means.

The term “distal end” according to instant invention shall mean the end of the device or a component of the device which is closest to the dispensing end of the device. The term “proximal end” according to instant invention shall mean the end of the device or a component of the device which is furthest away from the dispensing end of the device. Accordingly, the “distal direction” is the direction which is directed from the proximal end to the distal end of the device or a component of the device and the “proximal direction” is the direction which is directed from the distal end to the proximal end of the device or a component of the device.

According to another aspect of the invention, the invention concerns the use of a medication delivery device according to any of the embodiments described above. The use of the medication delivery device can be provided for dispensing a pharmaceutical formulation comprising an active compound selected from the group consisting of insulin, growth hormone, low molecular weight heparin, their analogues, and their derivatives.

According to the invention, also a method of manufacturing or assembling of a medication delivery device according to any of the embodiments described above.

The invention will now be described, by way of example only, with reference to the accompanying drawings, in which:

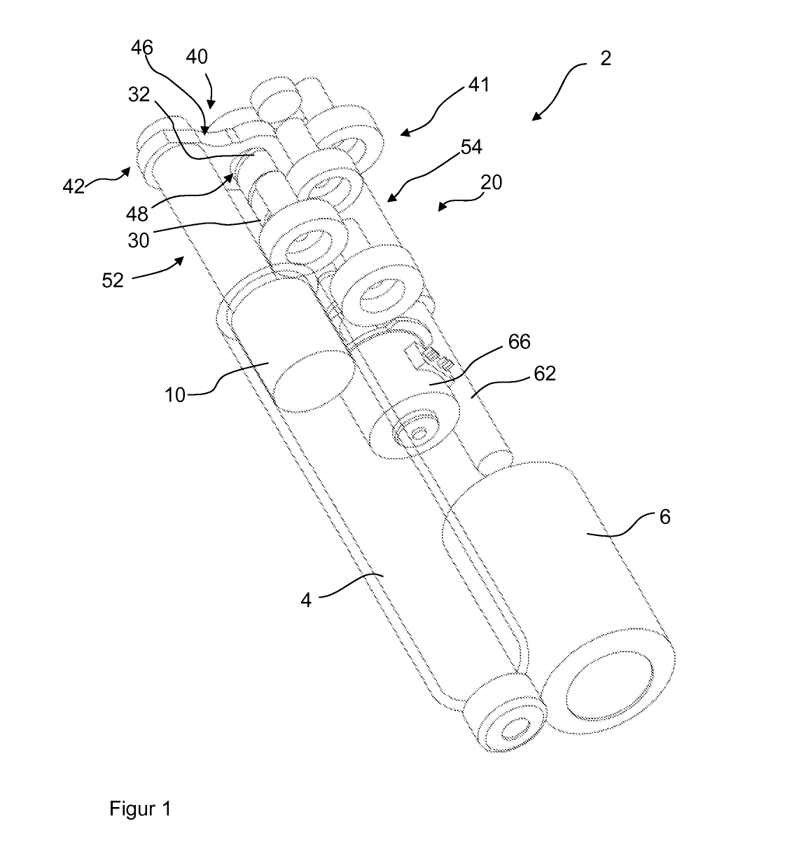

FIG. 1 shows a perspective view of a drive assembly according to the first aspect of the present invention in a first position together with a cartridge and power source;

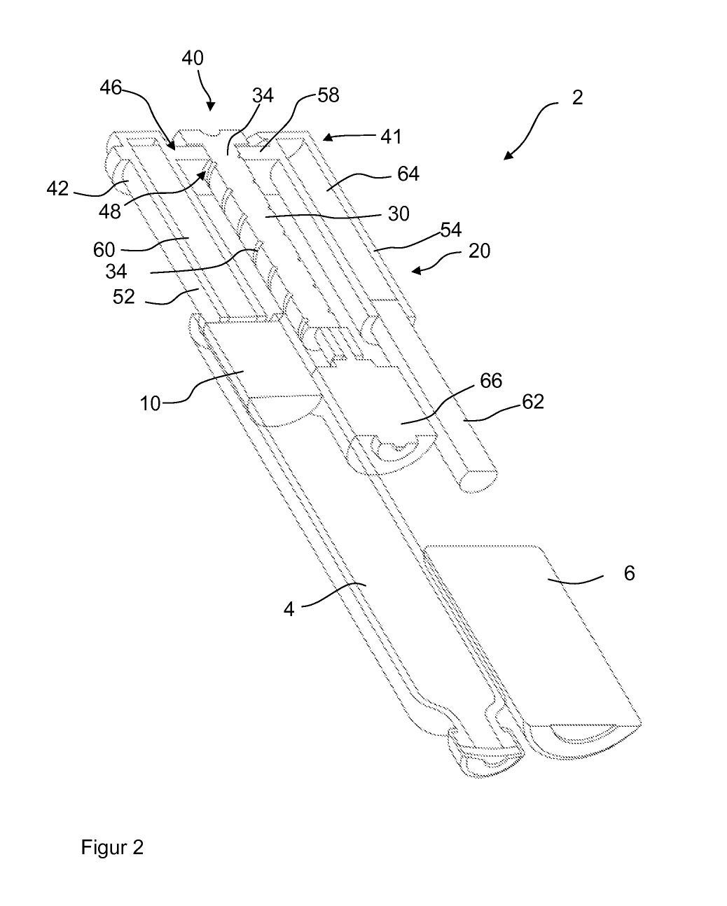

FIG. 2 shows a sectional view of the drive assembly of FIG. 1 in a first position together with a cartridge and power source;

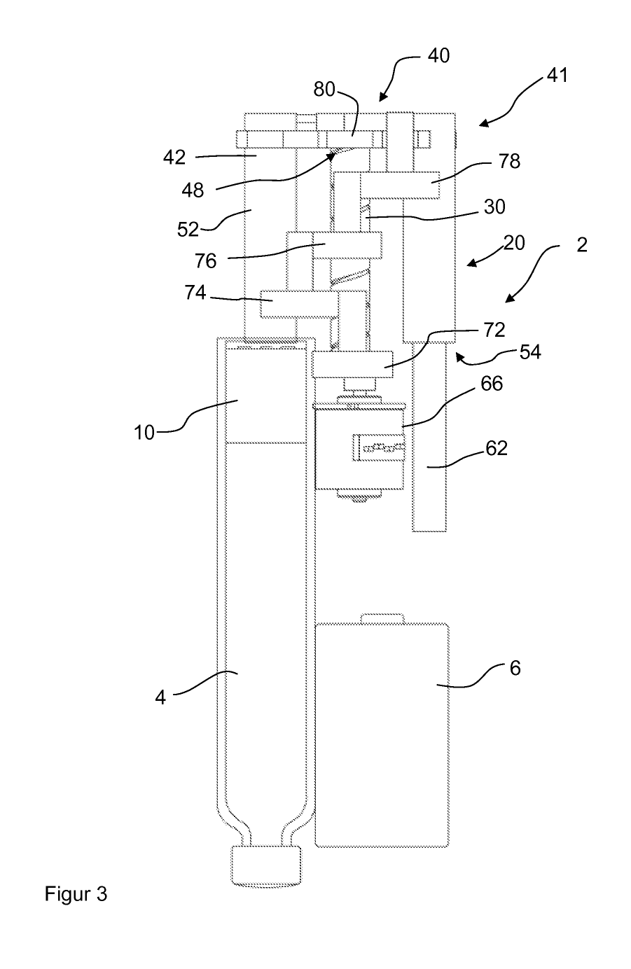

FIG. 3 shows a plan view of the arrangement shown in FIG. 1; and

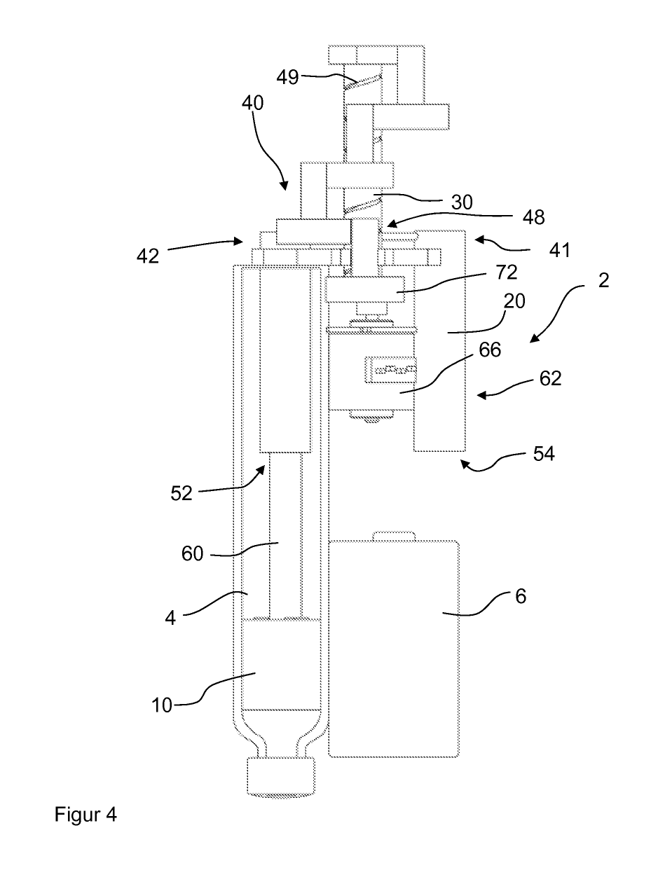

FIG. 4 shows a plan view of the arrangement shown in FIG. 1 with the drive assembly in a fully advanced position.

Referring first to FIGS. 1 and 2 there may be seen a perspective view of a drive assembly 2 in a first position together with a cartridge 4 and a power source 6. The cartridge 4 may be of the known kind comprising a cartridge piston 10 displaceable within a cylindrical body 8.

The power source 6 may conveniently comprise one or more batteries.

The drive assembly 2 comprises a drive means 20, an elongate member 30, and a housing 40. The elongate member 30 has a headed portion 32. The elongate member 30 is mounted for rotation about a longitudinal axis. The elongate member 30 is provided with a helical groove or helical rib 34 extending from the headed portion 32 along its length to form, a thread. The headed portion 32 is provided about a periphery with a plurality of gear teeth.

The housing 40 comprises a first elongate generally cylindrical portion 42, a second elongate generally cylindrical portion 41 and a cross member 46 joining a first end of the first cylindrical portion 42 to a first end of a second cylindrical portion 41. The first cylindrical portion 42 defines a first passage portion 52 therein and the second cylindrical portion 41 defines a second passage portion 54 therein. It may be seen that the first passage portion 52 and the second passage portion 54 extending substantially parallel to one another. The first passage portion 52 and the second passage portion 54 are connected by an intermediate passage portion 58.

The cross member 46 includes an opening 48 having a helical rib or a helical groove 49. The elongate member 30 extends through the opening 48 and is threadedly connected to the housing 40 thorough the opening 48.

In an alternative embodiment (not shown) the elongate member may be provided with a rib to match a groove formed in the cross member opening 48.

A piston member 60, and in particular a first piston member 60, is displaceably located within a second end of the first passage portion 52. An adjusting means, according to the figures in the form of a second piston member 62 is displaceably located within a second end of the second passage portion 54. The second piston member, 62, is fixedly located with respect to the elongate member 30.

It may be seen that a cavity 64 is defined by an inner surface of the housing 40, a first end of the first piston member 60 and a first end of the second piston member 62. The cavity 64 is filled with an incompressible medium (such as a liquid).

The drive means 20 comprises a motor 66 and a drive train. Conveniently, the motor 66 may comprise a stepper motor. In the illustrated embodiment (FIG. 3), the drive train comprises five gear elements. A first gear element 72 is driven from the motor 66 and drives a second gear element 74. The second gear element 74 drives a third gear element 76. The third gear element 76 drives a fourth gear element 78. The fourth gear element 78 in turn drives a fifth gear element 80. The fifth gear element 80 drives the elongate member 30 through the gear teeth provided on the headed portion 32 of the elongate member 30. The gear train may be so constructed to provide a suitable gear ratio between the motor 66 and the elongate member 30.

The motor 66 may conveniently be controlled by a microprocessor (not shown) in response to user input.

On receipt of a suitable command the motor 66 is actuated to drive the gear train and so the elongate member 30. On rotation of the elongate member 30, the housing 40 is induced to precess along the elongate member 30. Since the second piston member 62 is fixed, there is relative movement between the second piston member 62 and the housing 40. This has the result that the second piston member 62 fills a greater proportion of the second passage portion 54. Since the cavity 64 is filled with an incompressible medium, the first piston member 60 is advanced out of the first passage portion 52 to displace the cartridge piston 10 within the cartridge body 8 to cause medical product to be expelled through a needle unit (not shown).

It is an advantage that the compact arrangement of this construction enables a pen-type injector of compact length to be produced.

The term “drug” or “medicinal product”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(02)25, Asp28] Exendin-4(1-39),

des Pro36 [Met(O)14 Trp(02)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(02)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

1. A drive assembly for a pen-type injector, comprising:
a guiding means having a first and a second end,
a drive means,
an elongate member coupled to the guiding means and adapted to be driven by the drive means for displacing the guiding means with respect to the drive means, a piston member displaceably disposed at the first end of the guiding means and
an adjusting member displaceably disposed at the second end of the guiding means,
wherein the drive means is fixedly located with respect to the adjusting member, and
wherein the piston member and the adjusting member are coupled such that the sum of the distance between the piston member and the guiding means and the distance between the adjusting means and the guiding means remains constant, wherein the guiding means is formed as a U-shaped device, characterised in that the elongate member is adapted to be rotated by the drive means and wherein the guiding means comprises an opening, the opening and the elongate member forming a threaded connection between the U-shaped guiding means and the elongate member, rotation of the elongate member causing the guiding means to process along the elongate member, wherein the adjusting member is fixedly located with respect to the elongate member and, wherein the guiding means forms a housing with a passageway, wherein the piston member is displaceable located within a first end of the passageway, wherein the adjusting member is formed as a second piston member and is displaceable located within a second end of the passageway.

2. The drive assembly according to claim 1, wherein the housing is filled with an incompressible medium so that the housing, the incompressible medium and the first piston member and the second piston member are configured such that a displacement of the second piston member with regard to the passageway causes a displacement of the first piston member.

3. The drive assembly according to claim 2, wherein the passageway defines a first passage portion, a second passage portion extending parallel to the first passage portion and an intermediate passage portion connecting the first passage portion to the second passage portion.

4. The drive assembly according to claim 1, in which the drive means comprises a motor and in which the drive train comprises a gear train.

5. The drive assembly according to claim 1, in which the elongate member is provided with a helical rib to engage a helical thread provided in an opening in guiding means.

6. The drive assembly according to claim 1, in which the drive means comprises a motor drivingly connected to a gear train for driving the elongate member.

7. The drive assembly according to claim 6, in which the motor is a stepper motor.

8. The drive assembly according to claim 7, comprising a micro-processor configured to control the motor.

9. A pen-type injector comprising a main housing, and a drive assembly according to claim 1 and a power source for supplying the drive mechanism, wherein the guiding means is moveably integrated within the main housing.

10. The pen-type injector according to claim 9, wherein the injector comprises a replaceable cartridge, wherein the cartridge can be fixedly located with respect to the elongate member, wherein the guiding means is disposed displaceable with regard to the cartridge and wherein the guiding means is configured such that at least in an extended position of the same the first end of the guiding means extends into one end of the cartridge for dispensing a medium contained in the cartridge when the guiding means is moved into a dispensing direction.
